(19) United States
(12) Patent Application Publication
(10) Pub. No.: US 2022/0183624 A1
(43) Pub. Date: Jun. 16, 2022

(54) Dental Sensor For The Intraoral Region

(71) Applicant: Ivoclar Vivadent AG, Schaan (LI)

(72) Inventors: Christian Niedrig, Rüthi (CH); Tatiana Glebova, Buchs (CH); Jonas Reinhardt, Igis (CH)

(21) Appl. No.: 17/541,655

(22) Filed: Dec. 3, 2021

(30) Foreign Application Priority Data
Dec. 16, 2020 (EP) 20214651.0

(51) Int. Cl. A61B 5/00; B29L 31/00; B29C 35/02

(52) U.S. Cl. CPC A61B 5/682; A61B 2562/16; B29L 2031/753; B29C 35/02; B29L 2031/752

(57) ABSTRACT

The present invention relates to a dental sensor (100) for an intraoral region, having a region (101) of plastic material (103) for molding a tooth region (105) during insertion of the dental sensor (100), which is curable after molding.

### CROSS-REFERENCE TO RELATED APPLICATIONS

This application claims priority to European Patent Application No. 20214651.0 filed on Dec. 16, 2020, the disclosure of which is incorporated herein by reference in its entirety.

### TECHNICAL FIELD

The present invention relates to a dental sensor for an intraoral region and a method of inserting a dental sensor.

### BACKGROUND

Sensor housings that are not anatomically correct for intraoral use offer poor wearing comfort, are unnecessarily disruptive during food intake and are not adequately secured, as the natural shape of the dental arch cannot be used for additional adhesion. In addition, there is a risk that the dental sensor will detach and be swallowed when worn.

### SUMMARY

It is the technical aim of the present invention to provide a dental sensor that can be attached in a simple and quick manner by a user in the intraoral space.

This problem is solved by subject-matter according to the independent claims. Technically advantageous embodiments are the subject of the dependent claims, the description, and the drawings.

According to a first aspect, the technical problem is solved by a dental sensor for an intraoral region, with an attachment region made of plastic material for molding an oral region during insertion of the dental sensor, which is curable after molding. Curing may be performed inside or outside the oral cavity, i.e., intraorally or extraorally. The oral region may include a tooth region with one or more teeth, which may optionally be in contact with the gums.

Due to the shaping of the plastic material, the dental sensor can be quickly and individually adapted to the spatial conditions in the intraoral space. As a result, the position of the dental sensor can be better set. For example, shaping can be performed directly by a dentist in just a few minutes. Fastening by means of anatomically correct shaping means that there is no need to resort to chemical adhesion, which is costly to remove and damaging to the teeth.

In a technically advantageous embodiment of the dental sensor, the plastic material is curable by means of light, electromagnetic radiation, or heat. The plastic material can also be cured by drying, by a chemical reaction with water (hydration) or by addition curing. This has the technical advantage, for example, that curing can be initiated specifically after the impression has been taken.

In a further technically advantageous embodiment of the dental sensor, the plastic material comprises a curable polymer. This provides, for example, the technical advantage of using particularly suitable materials that cure quickly. The plastic material can retain a residual flexibility or residual elasticity after curing, so that it can be removed more easily, such as by using silicone-type materials. This provides the technical advantage, for example, that the dental sensor can be removed even if it becomes wedged, such as when the dental sensor is attached to a tooth space.

In a further technically advantageous embodiment of the dental sensor, the plastic material is arranged on a sensor housing of the dental sensor. This has, for example, the technical advantage that the sensor housing can be attached to the tooth region.

In a further technically advantageous embodiment of the dental sensor, a connection between the attachment region and the dental sensor or the sensor housing is established by means of a positively configured connecting means. The positively configured connecting means comprises, for example, one or more protruding studs or anchors that connect to the plastic material. In the method, the plastic material deforms, shapes, or presses around this structure. This has, for example, the technical advantage that a reliable connection can be achieved between the attachment region and the dental sensor.

In a further technically advantageous embodiment of the dental sensor, the sensor housing comprises a transparent or heat-conducting material and the transparent or heat-conducting material is in contact with the plastic material. This provides, for example, the technical advantage that light or heat can be introduced to the plastic material and the plastic material can be cured efficiently.

In a further technically advantageous embodiment of the dental sensor, the dental sensor comprises a light exposure device or a heating device for the plastic material. This has the technical advantage, for example, that the plastic material can be cured directly by the dental sensor.

In a further technically advantageous embodiment of the dental sensor, the light exposure device or the heating device such as a curing light can be activated by a user. This has, for example, the technical advantage that the user can control the curing of the plastic material.

In a further technically advantageous embodiment of the dental sensor, the activation of the light exposure device or the heating device is wireless. The activation of the light exposure device or the heating device can be carried out, for example, via W-Lan, NFC, or Bluetooth by means of a cell phone. This has the technical advantage, for example, that no actuation actions are required in the intraoral space.

In a further technically advantageous embodiment of the dental sensor, the attachment region, the dental sensor and/or the sensor housing comprises a prefabricated through opening and/or one or more channels. This provides, for example, the technical advantage that fluid from the tooth can be fed directly to a sensor that analyzes the fluid.

In a further technically advantageous embodiment of the dental sensor, the region around the through opening is not curable. This has the technical advantage, for example, that the dental sensor can be easily removed after curing.

In a further technically advantageous embodiment of the dental sensor, the plastic material is anatomically preformed. The anatomical preforming can, for example, comprise corresponding tooth-shaped protrusions for individual teeth. This has the technical advantage, for example, that the dental sensor is better shaped to the tooth region.

In a further technically advantageous embodiment of the dental sensor, a release layer is arranged on the plastic material for detaching the plastic material from the tooth region. This has the technical advantage, for example, that the dental sensor can be easily removed after the impression has been taken.

US 20080026344, 20210346690, 20210353180, 20210315460, and 20210282650 are directed to sensors for use in the oral cavity and are hereby incorporated by reference in their entirety.

According to a second aspect, the technical problem is solved by a method for inserting a dental sensor for an intraoral region, comprising the steps of molding a tooth region during insertion of the dental sensor by means of a plastic material; and curing the plastic material after the molding. Thereby, the same technical advantages are achieved as by the method according to the first aspect.

In a technically advantageous embodiment of the method, curing takes place by means of light, electromagnetic radiation, or heat. This also has the technical advantage, for example, that the curing can be initiated specifically after the impression has been taken.

### BRIEF DESCRIPTION OF THE FIGURES

Examples of embodiments of the invention are shown in the drawings and are described in more detail below.

It shows:

FIG. 1 a schematic side view of a dental sensor;

FIG. 2 a schematic top view of the dental sensor;

FIG. 3 a schematic view through the dental sensor;

FIG. 4 a schematic view through the dental sensor and/or attachment region with different channels; and

FIG. 5 a block diagram of a method for inserting a dental sensor.

### DETAILED DESCRIPTION

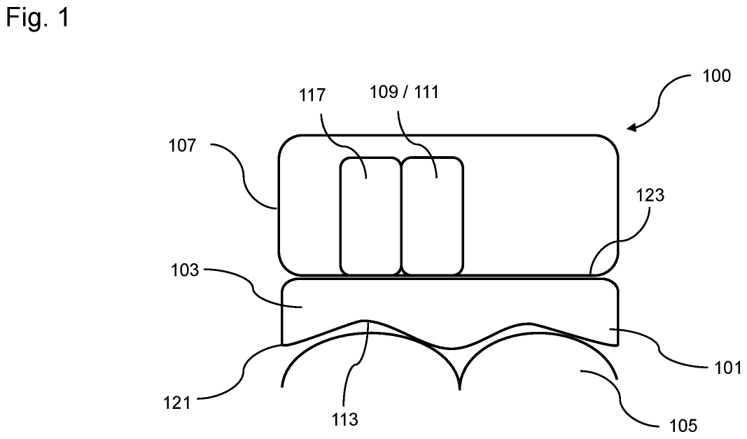

FIG. 1 shows a schematic side view of a dental sensor 100. The dental sensor 100 comprises a sensor housing 107 in which evaluation electronics 117 and a sensor unit are arranged. The evaluation electronics 117 and the sensor unit are together suitable for autonomously performing measurements of certain physical parameters on the tooth 105. The sensor housing 107 is made of plastic, for example, in a standard form.

The sensor housing 107 has a flat or approximately anatomically preformed tooth-facing contact surface 123 covered with a sheet-like layer of a moldable and plastic material 103. The plastic material 103 forms an attachment portion 101 for attaching the dental sensor 100 in the oral region. A layer thickness of the plastic material 103 is, for example, 1 mm to 10 mm. Suitable production-side structuring of the contact surface 123 of the sensor housing 107 or anchoring of the plastic material 103 through openings in the sensor housing 107 can be provided for fastening the plastic material 103.

An anatomically shaped contact surface 123 of the sensor housing 107 or in the plastic material 103 has the advantage of improving the wearing comfort of the dental sensor 100 and making it less distracting during food intake. The natural shape of the dental arch can be used for additional adhesion of the dental sensor 100.

The plastic and/or deformable material 103 deforms when pressed onto the tooth region 105, which may comprise one or more teeth.

In this way, a spatial impression of the tooth region 105 is obtained by the plastic material 103. Subsequently, the plastic material 103 is cured so that it loses its deformability. This can be achieved, for example, by means of irradiation with UV light, blue light, in contact with oxygen or saliva or by heat.

It would also be conceivable to use a material that is initially activated and then hardens over time at the tooth region 105. Preferably, the plastic material 103 is prefabricated so that the time required to attach the dental sensor 100 is reduced and potential sources of error during processing are eliminated.

U.S. Pat. Nos. 11,142,592, 11,078,303, 10,426,712, 10,932,995, 9,833,389, 8,889,196, 8,436,070 are directed to curable dental compositions, which are hereby incorporated by reference in their entirety.

The plastic material 103 comprises, for example, a hydrophilic vinyl polysiloxane impression compound or a polymer based on methacrylates and various fillers bonded to silanes, such as a light-curable nanohybrid composite.

Plastic material 103 comprises, for example, a monomer matrix composed of dimethacrylates (17-18 wt %). Fillers include, for example, barium glass, ytterbium trifluoride, and/or various oxides and copolymers (82-83 wt %). Additives, initiators, stabilizers, and pigments may be additional ingredients (<1.0 wt %). The total amount of inorganic fillers is, for example, between 53 and 80 vol %. The particle sizes of the inorganic fillers are, for example, between 40 nm and 3 μm.

The plastic material 103 may have antibiotic properties, such as through an incorporation of silver particles, copper particles, or a mixture with chlorhexidine and chloroxylenol. In addition, the plastic material may comprise antibiotics, such as penicillin, clindamycin, erythromycin, cefadroxil, metronidazole, and/or tetracyclines. The plastic material 103 may also be supplemented by a silicone or a plasticine can be formed.

For example, the dental sensor 100 may include an electronic light exposure device 109 disposed inside the sensor housing 107. The light exposure device 109 emits light by means of a light emitting diode, which causes the light-curing plastic material 103 to cure. In this case, the sensor housing 107 is formed of, for example, an optically transparent material that is in contact with the plastic material 103. This allows light for curing from an interior of the dental sensor 100 to pass through the sensor housing 107 to the plastic material 103 and cure it. However, a chemical light exposure device 109 based on the principle of chemiluminescence may also be provided, which is activated once and emits chemically generated light. The chemical light exposure device 109 may be formed, for example, by a luminescent rod insertable into the sensor housing 107.

However, the dental sensor 100 may also include a heating device 111 disposed inside the sensor housing 107. The heating device 111 emits heat by means of a heating coil, for example, which results in curing of the thermosetting plastic material 103. In this case, a metal is provided between the heating device 111 as a heat-conducting material between the heating device 111 and the thermosetting plastic material 103. The heat-conducting material allows the generated heat to be effectively supplied to the thermosetting plastic material 103 to cure it. However, a chemical heating device 111 may also be provided, which is activated once and emits chemically generated heat.

The light exposure device 109 or the heating device 111 can be manually activated by operating a switch or push button on the sensor housing so that they illuminate or heat for a predetermined time period. At the end of this time period, the plastic material 103 is cured.

However, the light exposure device 109 or the heating device 111 can also be activated wirelessly via radio, for example via WLAN, NFC or Bluetooth using a cell phone or tablet PC. In this case, a corresponding interface is implemented in the evaluation electronics 117 via which the light exposure device 109 or heating device 111 can be controlled.

In addition, a release layer 121 may be provided as a release agent to facilitate detachment of the dental sensor 100 from the tooth or tooth region 105 after curing. The release layer 121 is additionally arranged on the plastic material 103 and prevents direct contact between the tooth 105 and the plastic material 103. This release layer 121 may, for example, be a thin film of grease or oil or formed by a protective membrane of rubber, Teflon, or latex. In this case, the dental sensor 100 can be removed again without leaving any residue, even without cured plastic material 103.

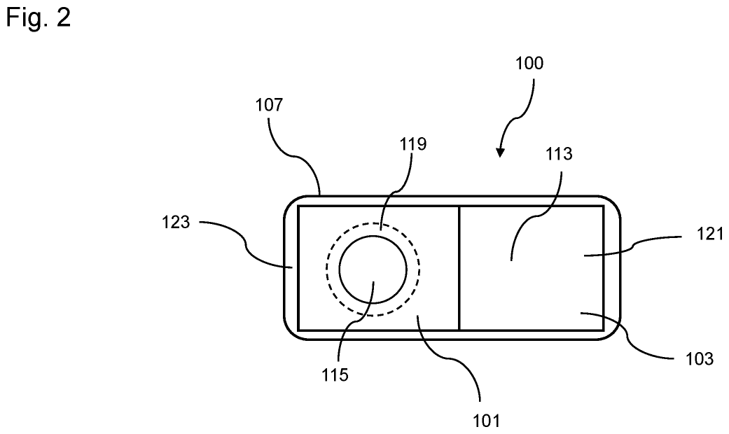

FIG. 2 shows a schematic top view of the dental sensor 100 and the plastic material 103. On the side of the plastic material 103 facing the tooth 105, a recess or through-opening 115 may already be provided in a predetermined shape on the production side, e.g. for a built-in sensor unit or any saliva channels for better supply of saliva from the tooth region 105 to the sensor unit inside the sensor housing 107 during intraoral use.

For example, the sensor unit may be a sensor for measuring a pH, an ethanol concentration, a lactate concentration, a cortisol concentration, a glucose concentration, an ion concentration, for measuring sound waves during biting together, and or a sensor for measuring a temperature. In general, the sensor unit within the dental sensor 100 can be used to perform intraoral measurement of various characteristics over an extended period of time.

This provided recess or through-opening 115 may, for example, be made of plastic material 103 that is not curable in a surrounding region 119 around the through-opening 115. In the case of a light-curable material 103, this can be done, for example, by not introducing photoinitiators into this surrounding region 119. Thus, this surrounding region 119 does not cure after the impression is taken under the influence of light and can be removed relatively easily. The surrounding region 119 may also comprise a water-soluble material, such as sugar, cornstarch, or a water-soluble filament.

An anatomical adaptation of the dental sensor 100 can be achieved, for example, by tooth-shaped or concave protrusions 113 in the contact surface 123 of the dental sensor 100, which correspond at least approximately to the tooth region 105. In this way, the dental sensor 100 can be arranged closer to the tooth region 105. Not only the contact surface 123, but also the plastic material 103 can be anatomically preformed with corresponding tooth-shaped or concave protrusions 113.

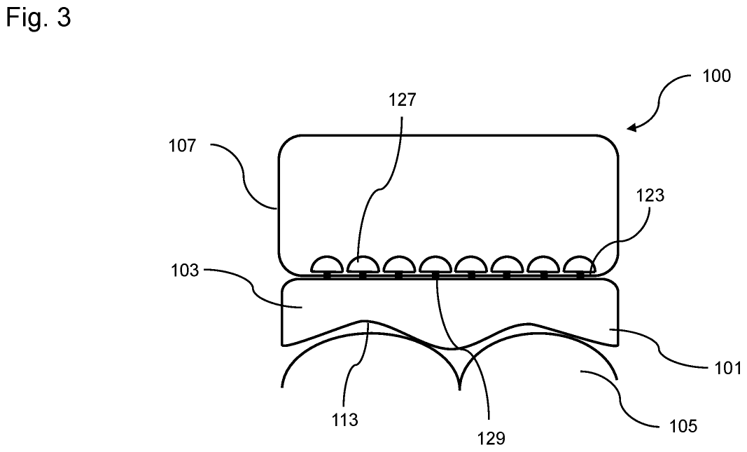

FIG. 3 shows a further schematic view through the dental sensor 100. The attachment region 101 with the plastic material 103 is attached through a plurality of openings 129 disposed in the sensor housing 107. During attachment, the plastic and uncured material 103 is partially forced through the openings 129 in the housing wall and then flattened on the inside of the sensor housing 107. Thereafter, an interior curing step may be performed to solidify the plated structures 127. In this way, the plastic material 103 clings to the sensor housing 107 and cannot come loose. The plastic material 101 holds itself through the mushroom-shaped structures 127 after curing.

Another means of attachment can be achieved by microstructuring the contact surface 123, such as by simple grinding.

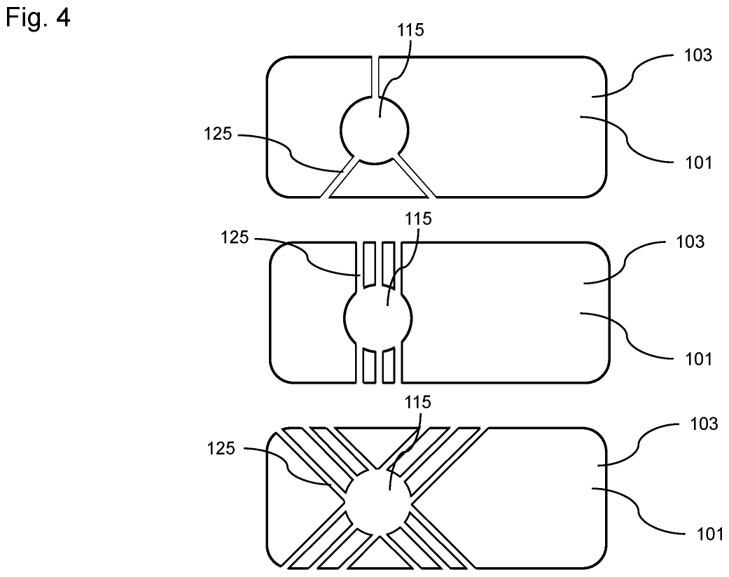

FIG. 4 shows a schematic view of the attachment region 101, the dental sensor 100 and/or the dental sensor housing 107 with different channels 125. Furthermore, the through opening 115 for enabling measurement by sensor unit may be provided in the plastic material 103. The through opening 115 forms an additional measurement region for the sensor unit in addition to the channels. For example, the through opening 115 may be made of plastic material 103 that is non-hardenable in a surrounding region 119 around the through opening 115.

The channels 125 are formed in the plastic material 103 by depressions and are used to conduct or carry liquid (saliva) to a sensor unit or to allow air exchange and ventilation of the measuring region. The channels 125 may be arranged in a horizontal, diagonal, or vertical direction.

The channels 125 can be formed, for example, by not introducing a photo initiator into the light-curable plastic material 103 at the intended locations of the channels 125. After light curing, the non-curable plastic material 103 without the photoinitiators can be removed at these locations, such as with a water jet or a spatula, to obtain the channels 125 in the attachment region 101.

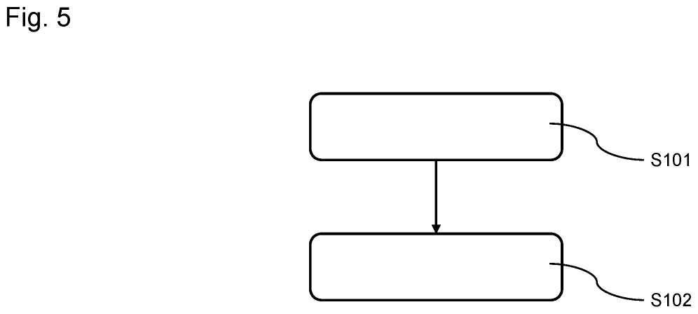

FIG. 5 shows a block diagram of a method for inserting a dental sensor 100. In step S101, a plastic material 103 is molded in the form of the tooth region 105 when the dental sensor 100 is inserted by pressing it onto the tooth region. The plastic material 103 thereby adapts to the shape of the tooth 105. Subsequently, in step S102, the plastic material 103 is cured after molding. Depending on the embodiment, the dental sensor 100 can thereby remain in the oral cavity or be cured outside the oral cavity. In this way, the attachment region 101 can be attached to a tooth 105.

The individual shaping can be accomplished in a few time-saving steps directly in the patient's mouth in just one session. Such an anatomically individualized shaping of an intraoral sensor carrier on the patient makes the dental sensor 100 suitable for permanent wear. It can be worn during food intake, during conversations and during sleep. Compared to individual 3D printing by means of impression taking, scanning and production in the dental laboratory, the shaping and adaptation of the dental sensor 100 can be carried out directly by the dentist in a short time. In addition, sensor positioning close to the tooth is made possible, as the dental sensor 100 is anatomically individually adapted to the patient.

All features explained and shown in connection with individual embodiments of the invention may be provided in different combinations in the subject matter of the invention to simultaneously realize their beneficial effects.

All method steps can be implemented by devices which are suitable for executing the respective method step. All functions that are executed by objective features can be a method step of a method.

The scope of protection of the present invention is given by the claims and is not limited by the features explained in the description or shown in the figures.

### REFERENCE LIST

100 Dental sensor

101 Attachment portion

103 Plastic material

105 Tooth region/tooth

107 Sensor housing

109 Light exposure device

111 Heating device

113 Protrusion

115 Through opening

117 Evaluation electronics

119 Surrounding region

121 Release layer

123 Contact surface

125 Channel

127 Structure

129 Opening

1. A dental sensor (100) for an intraoral region, comprising
an attachment portion (101) of plastic material (103) for molding onto an oral region (105) during insertion of the dental sensor (100), which plastic material is curable after molding.

2. The dental sensor (100) according to claim 1,
wherein the plastic material (103) is curable by light, electromagnetic radiation, or heat.

3. The dental sensor (100) according to claim 1,
wherein the plastic material (103) comprises a curable polymer.

4. The dental sensor (100) according to claim 1,
wherein the plastic material (103) is disposed on a sensor housing (107) of the dental sensor (100).

5. The dental sensor (100) according to claim 1,
wherein a connection between the attachment portion (101) and the dental sensor (100) or the sensor housing (107) is established by a positively configured connection.

6. The dental sensor (100) of claim 4,
wherein the sensor housing (107) comprises a transparent or thermally conductive material and the transparent or thermally conductive material is in contact with the plastic material.

7. The dental sensor (100) according to claim 1,
wherein the dental sensor (100) comprises a light exposure device (109) or a heating device (111) for the plastic material.

8. The dental sensor (100) according to claim 7,
wherein the light exposure device (109) or the heating device (111) is activatable by a user.

9. The dental sensor (100) according to claim 8,
wherein the activation of the light exposure device (109) or the heating device (111) is wireless.

10. The dental sensor (100) according to claim 4,
wherein the attachment portion (101), the dental sensor (100) and/or the sensor housing (107) comprises a prefabricated through opening (115) and/or one or more channels (125).

11. The dental sensor (100) according to claim 10, comprising
a surrounding region (119) around the through opening (115),
wherein the surrounding region (119) of the plastic material (103) around the through opening (115) is not curable.

12. The dental sensor (100) according to claim 1,
wherein the plastic material (103) is anatomically preformed.

13. The dental sensor (100) according to claim 1,
wherein a release layer (121) is disposed on the plastic material (103) for releasing the plastic material (103) from the mouth region (103).

14. A method of inserting a dental sensor (100) for an intraoral region, comprising
molding (S101) onto an oral region (105) during insertion of the dental sensor (100) using a plastic material (103); and
curing (S102) of the plastic material (103) after molding.

15. The method according to claim 14,
wherein the curing is performed by light, electromagnetic radiation, or heat.
